(19) United States
(12) Patent Application Publication
(10) Pub. No.: US 2004/0192986 A1
(43) Pub. Date: Sep. 30, 2004

(54) Removal of sulfur compounds

(76) Inventors: Albert H. Wu, Medford, NJ (US); James E. Thompson, Pittsburgh, PA (US)

Correspondence Address: BUCHANAN INGERSOLL PC, 1835 MARKET STREET, 14TH FLOOR, PHILADELPHIA, PA, 19103-2985, US

(21) Appl. No.: 10/401,011

(22) Filed: Mar. 26, 2003

(51) Int. Cl. C07C 2/64; C07C 15/067

(52) U.S. Cl. 585/449

(57) ABSTRACT

Method of removing sulfur compounds from olefinic hydrocarbons by contacting with nickel-based sorbents. The method provides for the removal of heavy sulfur compounds including disulfides and mercaptans from pre-treated olefinic hydrocarbon feedstocks used in cumene synthesis to improve the life and efficiency of catalysts used in the cumene manufacturing process.

### FIELD OF THE INVENTION

This invention relates in general to methods of treating hydrocarbon feedstocks to remove sulfur. More specifically, this invention relates to a method of removing sulfur containing compounds by contacting olefinic hydrocarbon feedstocks with nickel-based sorbents. More specifically, this invention relates to a method of removing sulfur containing compounds from pre-treated propylene feedstocks that are contacted with pre-treated benzene feedstocks used in cumene synthesis.

### BACKGROUND OF THE INVENTION

Zeolite catalysts, particularly versions of the present generation of increased activity catalysts, are very effective for catalyzing industrial-scale cumene synthesis via benzene/propylene reactions. While their catalytic efficacy is impressive, most zeolites are highly susceptible to nitrogen-, sulfur-, and oxygen-containing contaminants and olefinic contaminants. In U.S. Pat. No. 6,355,851 to Wu et al., we disclosed a method for improving the activity and lifetime of zeolite catalysts by pre-treating benzene and propylene feedstocks used in cumene synthesis. The method disclosed the removal of light sulfur compounds such as hydrogen sulfide and carbonyl sulfide. However, it was discovered that during pre-treatment of olefinic hydrocarbons such as propylene, some carbonyl sulfide is transformed into mercaptans. Further, the '851 method is ineffective to remove higher molecular weight sulfur compounds. Non-limiting examples of higher molecular weight sulfur compounds are disulfides and mercaptans. It is known that nickel-based adsorbents can be used to remove sulfur-bearing species and arsine compounds from propylene prior to polymerization, as disclosed in “Adsorbent key to polypropylene catalyst activity”, Oil & Gas Journal, Oct. 10, 1994. Therefore it would be desirable to provide a method for removing contaminants including heavy organic sulfur compounds naturally present or converted during pretreatment of olefinic hydrocarbons to improve the activity and lifetime of a zeolite catalyst such as that would be used in cumene synthesis.

It is also known that spent nickel-based absorbents used to remove carbonyl sulfide, hydrogen sulfide and arsine from hydrocarbons can be reactivated. U.S. Pat. No. 5,130,282 to Bodart et al. discloses such a method. However, that method is limited to lighter sulfur compounds, and since a portion of the carbonyl sulfide present in olefinic hydrocarbon feedstocks converts to heavier sulfur compounds, a method of regenerating absorbents used to remove heavier sulfur compounds such as mercaptans and dimethyl sulfide would be desirable.

### SUMMARY

Briefly, the invention relates to a method for treating olefinic hydrocarbon feedstocks to remove sulfur compounds. The method provides for contacting olefinic hydrocarbon feedstocks with a nickel-based sorbent to remove heavy organic sulfur compounds.

In one embodiment of the present invention, an olefinic hydrocarbon feedstock such as propylene is treated through contact with a nickel-based sorbent on a porous support. In this example, the nickel-based sorbent adsorbs heavy organic sulfur compounds such as mercaptans and disulfides.

According to another embodiment of the present invention, a propylene feedstock is pre-treated for cumene synthesis. In this example, propylene is treated by contacting the propylene feedstock with a regenerable nickel-based sorbent to remove heavy organic sulfur compounds that are naturally present or converted to heavy organic sulfur compounds during pre-treatment with alumina. Regeneration occurs by bubbling a hot non-oxidizing gas such as nitrogen or hydrogen gas through the porous support to desorb heavy organic sulfur compounds removed during treatment of an olefinic hydrocarbon feedstock. The pre-treated propylene feedstock is combined with a benzene feedstock to synthesize cumene. Removing heavy organic sulfur compounds increases the efficiency and effectiveness of zeolite catalysts used in cumene synthesis.

### BRIEF DESCRIPTION OF DRAWINGS

FIG. 1 is a schematic depiction of a cumene synthesis unit which illustrates the nickel-based sorbent contact bed.

### DETAILED DESCRIPTION OF THE INVENTION

The present invention provides a method for removing sulfur containing compounds from olefinic hydrocarbon feedstocks. According to one embodiment of the present invention, an olefinic hydrocarbon is contacted with a nickel-based sorbent to remove organic sulfur compounds. The organic sulfur compounds are either naturally occurring in the olefinic hydrocarbon stream or occurring as the result of conversion of approximately 50% or more of other sulfur compounds such as carbonyl sulfide in the olefinic hydrocarbon during pre-treatment to heavier sulfur compounds such as disulfides and mercaptans. The olefinic hydrocarbon is preferably a C₃ to C₅ hydrocarbon, more preferably propylene. Any nickel type catalyst, preferably a reduced nickel catalyst may be used. A preferred example of a nickel catalyst is Engelhard's Prosorb or Süd-Chemie's TSR-11®.

The contacting of an olefinic hydrocarbon feedstock with a nickel-based sorbent may be accomplished by any means that enables sulfur removal from the feed. A preferred method is the use of granular absorptive materials in a packed bed, although a fluidized bed or other contacting means may be employed.

In addition to removal of heavy sulfur compounds, the present method also reduces arsine levels in olefinic hydrocarbon feedstocks.

The method of removing sulfur containing compounds from olefinic hydrocarbon feedstocks improves the life and efficiency of catalysts used in cumene synthesis. According to one embodiment of the invention, the olefinic hydrocarbon feedstock is preferably propylene, and the propylene feedstock is pre-treated by contact with at least one alumina to remove trace sodium compounds, moisture, and catalyst poisons.

According to another aspect of the present invention, the nickel-based sorbent is regenerable. Preferably, an inert gas is blown through the bed containing the nickel-based sorbent. More preferably, nitrogen or hydrogen is blown through the bed, at a temperature between 150° to 450° C., preferably 200° to 300° C., causing desorption of the heavy organic sulfur compounds from the porous bed material. The flow of inert gas is preferably such that the gaseous space velocity is approximately 200-300 liters/(liters per hour). The preferred operating pressure for regeneration is atmospheric pressure or slightly above. The duration of the contact with the inert gas is between 1 and 24 hours, preferably 24 hours. Though not wishing to be bound by a theory, it is postulated that the inert gas causes the chemical reduction of the sulfur compounds so that sulfur is carried out in the effluent gases.

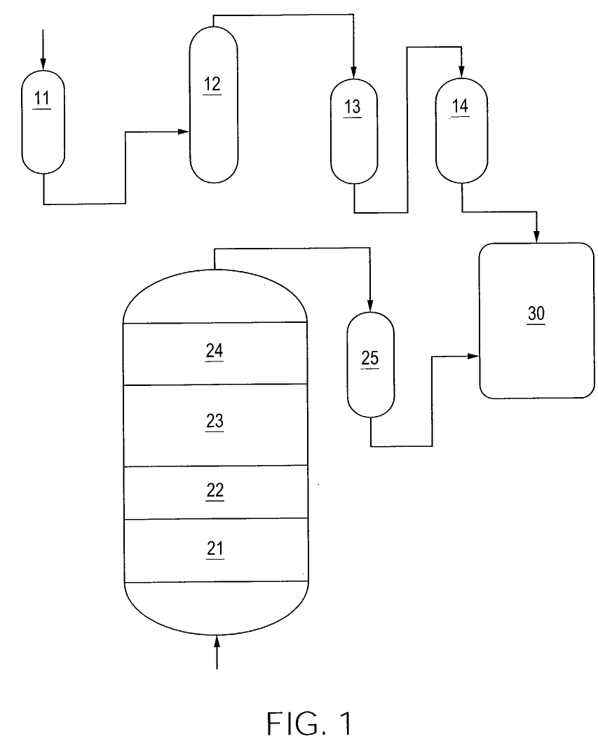

Referring now to FIG. 1, the schematic shown illustrates one embodiment of the present method incorporated into a cumene synthesis unit. Benzene feedstock containing olefinic contaminants is introduced into hot clay treater 11 containing a bed of hot clay to convert the olefinic contaminants into higher boiling polymers, alkylaromatics or other hydrocarbons. The benzene feed is then introduced into distillation column 12, where the higher boiling converted olefinic contaminants are separated and removed from the benzene feed. The benzene feed is then sequentially introduced into cold clay treaters 13 and 14, where nitrogen containing poisons are removed from the benzene feed through contact with ambient-temperature clays.

The propylene feedstock is introduced into a vertical treater housing multiple treatment stages 21, 22, 23, 24, and 25. The propylene feedstock is introduced into the bottom of the treater and is caused to flow upwardly to minimize the amount of moisture-and-contaminant-rich mist associated with the feedstock from entering the treater. The propylene feedstock first enters treatment stage 21, and there contacts a bed of alumina capable of removing trace sodium compounds and moisture from the feed. The feed then flows into adjacent treatment stage 22 which comprises a bed of a zeolite molecular sieve. The feed contacts the molecular sieve which removes additional moisture from the feed. Immediately downstream from treatment stage 22 is a two-stage modified alumina treatment stages 23 and 24. The first modified alumina bed removes nitrogen-, sulfur- and oxygen-based poisons from the feed. The second modified alumina bed selectively removes hydrogen sulfide and carbonyl sulfide from the propylene feed. Finally, the pre-treated propylene feedstock contacts sulfur/arsine removal treatment stage 25. In that stage, the feed contacts the nickel-based sorbent bed. The pre-treated benzene and propylene feeds are introduced into cumene synthesis reactor 30, which houses a zeolite catalyst.

### EXAMPLE

A nickel adsorbent bed using Engelhard CO2-Passivated Nickel Adsorbent was activated by heating to greater than 400° F. with hot nitrogen gas at approximately 50 GHSV at 400° F. and 1 atmosphere pressure. The bed was heated for approximately 24 hours. Activation is complete when the carbon dioxide concentration is less than 100 ppm at the adsorbent bed outlet. The bed was cooled down to room temperature with unheated nitrogen.

The adsorbent bed was operated at 400 psig and at room temperature. Liquid propylene doped with sulfur from methyl mercaptan was fed through the bed. The following chart and table illustrate the removal of sulfur from the feed stream before and after regeneration of the nickel based sorbent. Regeneration was performed after the total sulfur in the product was measured at 6.6 ppmw as indicated on the chart below, over 35 days after beginning the bed operation. The measurements presented on Table I and Chart I were taken every few days and correspond to the flow measured in liters through the absorbent bed, as indicated below.

TABLE I

| Cumulative Flow (L) | S in Feed Stream (ppm) | S in Product (ppm) |
|---|---|---|
| 394.9 | 11.3 | 0.13 |
| 2701.0 | 10.9 | 0.11 |
| 6921.9 | 14.1 | 0.7 |
| 9401.9 | 0.07 | 0.53 |
| 12398.3 | 25.3 | 1.2 |
| 12609.3 | 22.2 | 1.6 |
| 16641.9 | 19.6 | 7.9 |
| 17632.1 | 20.5 | 6.7 |
| 17821.4 | 20.8 | 5.5 |
| 18412.4 | 2.0 | 10.4 |
| 19182.7 | 2.4 | 4.3 |
| 21308.5 | 1.5 | 1.0 |
| 23941.0 | 1.7 | 0.62 |
| 25931.5 | 1.2 | 0.54 |
| 26437.8 | 1.6 | 0.54 |
| 26888.1 | 15.0 | 3.3 |
| 27949.9 | 17.0 | 6.4 |
| 28273.3 | 18.0 | 6.6 |
| Regeneration | | |
| 28817.8 | 10.0 | 0.18 |
| 29205.3 | 12.1 | 0.13 |

[image]

What is claimed is:

1. An alkylation process comprising:
treating an olefinic hydrocarbon feedstock to remove sulfur-containing compounds by contacting said olefinic hydrocarbon feedstock with a nickel-based sorbent, and
reacting said olefinic hydrocarbon feedstock with a benzene feedstock in the presence of a zeolite catalyst.

2. The method of claim 1, further comprising pre-treating said olefinic hydrocarbon feedstock by contacting said olefinic hydrocarbon feedstock with at least one alumina to remove sodium compounds, nitrogen-containing compounds, oxygen-containing compounds, and sulfur-containing compounds.

3. The method of claim 2, further comprising pre-treating said olefinic hydrocarbon feedstock by contacting said olefinic hydrocarbon feedstock with a molecular sieve to remove trace moisture.

4. The method of claim 1, further comprising pre-treating said benzene feedstock to remove catalyst poisons comprising olefinic compounds, said benzene feedstock pre-treatment comprising contacting said benzene feedstock with a clay to convert said olefinic compounds to polymers, alkylaromatics and other hydrocarbons having a higher boiling point than benzene, and distilling said benzene feedstock after contacting with said clay to separate said benzene feedstock from said polymers, alkylaromatics and other hydrocarbons having a higher boiling point than benzene.

5. The method of claim 4 wherein said clay is at a temperature within the range of about 200° to 500° F. and said benzene feedstock is under a pressure during said contacting with said clay to maintain said benzene feedstock in a liquid phase.

6. The method of claim 1, wherein said nickel-based sorbent comprises reduced nickel on a porous support.

7. The method of claim 1, wherein said olefinic hydrocarbon feedstock is propylene.

8. The method of claim 1, wherein said sulfur-containing compounds include high molecular weight sulfur-containing compounds.

9. The method of claim 8, wherein said high molecular weight sulfur-containing compounds include mercaptans and dimethyl sulfide.

10. A method for manufacturing cumene, said method comprising:
contacting a propylene feedstock with at least one alumina to remove sodium compounds, moisture, nitrogen-containing compounds, oxygen-containing compounds, and sulfur-containing compounds;
contacting said propylene feedstock with a nickel-based sorbent to remove sulfur-containing compounds;
contacting a benzene feedstock containing olefinic compounds with clay to convert said olefinic compounds to polymers, alkylaromatics and other hydrocarbons having a higher boiling point than benzene;
distilling said benzene feedstock to separate said benzene feedstock from said polymers, alkylaromatics, and other hydrocarbons having a higher boiling point than benzene, and reacting said propylene feedstock and said benzene feedstock in the presence of a zeolite catalyst.

11. The method of claim 10 further comprising contacting said propylene feedstock with a molecular sieve to remove moisture.

12. The method of claim 10, wherein said nickel is reduced nickel.

13. The method of claim 10, wherein said contacting with nickel-based sorbent is at a temperature of up to 200° F.

14. The method of claim 10, wherein said contacting with nickel-based sorbent is at a temperature of from 60° to 150° F.

15. A method of regenerating a nickel-based sorbent used to remove heavy sulfur compounds and arsenic from a pre-treated propylene feedstock to be reacted with a benzene feedstock in the presence of a zeolite catalyst wherein said propylene feedstock was pretreated to remove sodium compounds, moisture, nitrogen-containing compounds, oxygen-containing compounds and sulfur-containing compounds, said method comprising desorbing said heavy sulfur compounds from said nickel-based sorbent by contacting said nickel-based sorbent with an inert gas.

16. The method of claim 15 wherein said inert gas is nitrogen gas or hydrogen gas.

17. The method of claim 15 wherein said contacting with inert gas is at a temperature within the range of 200° to 300° C.

18. The method of claim 15 wherein said nickel is reduced nickel.
